(19) United States
(12) Patent Application Publication
(10) Pub. No.: US 2005/0070495 A1
(43) Pub. Date: Mar. 31, 2005

(54) Inhibition of DNA modulation caused by mutated p53

(76) Inventor: Wolfgang W. Deppert, Hamburg (DE)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/890,433

(22) Filed: Apr. 22, 2003

Related U.S. Application Data
(63) Continuation of application No. 09/600,981, filed on Oct. 4, 2000, now abandoned.

(30) Foreign Application Priority Data
Jan. 26, 1998 (DE) 19802792.3

(51) Int. Cl. A61K 48/00; C12Q 1/68; A61K 39/395; C12N 15/85

(52) U.S. Cl. 514/44; 435/455; 424/130.1

(57) ABSTRACT

The invention relates to a method for inhibiting DNA modulation caused by mut p53, comprising the inhibition of mut p53 bonding to DNA with strand separating potential. The invention also relates to a system for identifying substances suitable for said inhibition.

The present invention relates to a method of inhibiting the DNA modulation caused by mutated p53. The invention also relates to a system for identifying substances suitable for such an inhibition.

A protein referred to as p53 is present in cells. This protein is a tumor suppressor which is activated in the case of DNA damage. It then binds to promoters of target genes and activates the transcription thereof. As a result, growth stand-still of the cells and subsequent repair of DNA damage and death of the cells, respectively, is achieved.

As has been shown, p53 is mutated in many tumors. In this form, it often has no tumor-suppressor activity. It rather presents itself even as a protein which has oncogenic properties. Various experiments have been made to inhibit mutated p53 (hereinafter referred to as mut p53) as regards its oncogenic properties. However, these experiments did not yield satisfactory results.

Therefore, it is the object of the present invention to provide a product by means of which mut p53 can be investigated and optionally inhibited as regards its oncogenic properties.

According to the invention this is achieved by the subject matters defined in the claims.

Thus, the subject matter of the present invention relates to a method of inhibiting the DNA modulation caused by mut p53, comprising the inhibition of mut p53 bonding to DNA with strand separating potential. and a DNA with strand separating potential or a strand separation thereof. In particular, the DNA with strand separating potential may be one that comprises one or several copies of the sequence AATATATTT or a variation thereof. Besides, the DNA can also comprise further regions rich in AT. A DNA with strand separating potential is found preferably in MAR DNA.

The expression “bonding” comprises any kind and way by which mut p53 can bind to DNA with strand separating potential. In particular the bonding may be one where mut p53 binds directly to the DNA. The bonding may also be one where mut p53 binds indirectly, i.e. via other factors, such as proteins, to the DNA.

The expression “inhibition” comprises any kind and way by which mut p53 bonding to DNA with strand separating potential can be inhibited. The kind of inhibition will depend on whether mut p53 bonding to the DNA is direct or indirect. In the case of indirect bonding, i.e. via further factors, such as proteins, the inhibition may take place through substances which inhibit said further factors. It is also possible to add substances which inhibit mut p53. In the case of direct bonding of mut p53 to the DNA, it seems to be useful to employ substances inhibiting mut p53. Such substances may be e.g. those which inhibit a mutated core domain of p53 and/or a mutated C terminus of p53. Examples of such substances are the antibodies PAb 240 and PAb 421.

According to the invention a system for identifying substances is also provided which is suitable for inhibiting DNA modulation caused by mut p53. Such a system comprises mut p53 and a DNA with strand separating potential as well as optionally a substance whose inhibitory effect on mut p53 bonding to a DNA with strand separating potential is tested. As to individual components of the system the above explanations apply correspondingly. Furthermore, reference is made to the fact that a modulation of DNA and the inhibition thereof, respectively, can be determined by common methods. For example, DNA strand separation and complex formation, respectively, and the inhibitions thereof can be determined by an EMSA (“Electrophoretic Mobility Shift Assay”) test. For this purpose, it is an obvious thing to incubate mut p53 with labeled double-stranded DNA which has a strand separating potential and to separate the mixture electrophoretically so that the formation of single-stranded DNA and a complex thereof, respectively, and the inhibitions thereof become visible.

By means of the present invention it is possible to inhibit the DNA modulation caused by mut p53. Such a modulation can be a firm complex of mut p53 and a DNA with strand separating potential or a DNA strand separation thereof. The modulation of DNA plays a major part in many processes of the cell. For example, the DNA strand separation is an essential step for the expression of genes and the replication of DNA. The DNA strand separation is subject to strong control. This control is cancelled by mut p53. Thus, the way for the degeneration of the cell, i.e. for the tumor formation, has been paved.

By means of the present invention it is possible to take therapeutic steps in the case of diseases where mut p53 causes DNA modulation. Such diseases are in particular tumoral diseases. Furthermore, the present invention distinguishes itself in that it provides the possibility of identifying substances suitable for the inhibition of DNA modulation caused by mut p53, in particular in the case of tumors. Such substances also represent a subject matter of the present invention.

### BRIEF DESCRIPTION OF THE DRAWINGS

FIG. 1 shows the DNA strand separation caused by mut p53 in the case of MARI DNA,

FIG. 2 shows the DNA strand separation caused by mut p53 in the case of MARII DNA,

FIG. 3 shows the complex formation caused by mut p53 in the case of MARS DNA,

FIG. 4 shows the complex formation caused by mut p53 in the case of MAR7 and MAR8 DNAs, and

FIG. 5 shows the inhibition of the DNA strand separation caused by mut p53 in the case of MARI DNA.

The invention is explained by the below example.

### EXAMPLE

### Modulation of DNA by mut p53 and the Inhibition Thereof

The modulation of DNA is shown in the form of a DNA strand separation and a complex formation, respectively.

(a) Induction of DNA Strand Separation and a Complex Formation, Respectively, by mut p53

Two MAR regions in the form of oligonucleotides were drafted from the 997 bp XbaI IgE MAR fragment which lies in the enhancer region of the gene for the heavy immunoglobulin chain. These were MARI, i.e. the 3′-flanking region of the enhancer, and MARII, i.e. the 5′-flanking region of the enhancer. The oligonucleotides had the following sequences:

| | |
|---|---|
| MARI IgH enhancer | 5′- |
| | 3′-flanking region |
| | AGTGTCTTTAATTTCTAA- |
| | TATATTTAGAAAACTGC |
| MARII IgH enhancer | 5′- |
| 5′-flanking region | TTTTAACAATAATAAAT- |
| | TAAGTTTAAAATATTT- |
| | GCG-3′ |

MARI has the above indicated sequence AATATAATTT. MARII shows a variation of this sequence, the overall AT character not being modified.

Furthermore, oligonucleotides were drafted which have variations of MARI such that the overall AT character is reduced. These oligonucleotides had the following sequences:

| | |
|---|---|
| MAR6: | ACTATGCTT |
| MAR7: | GCTCTCTTT |

Furthermore, oligonucleotides were drafted which have the sequence of MARI together with the adjacent “GC clamps”. The overall AT character was reduced by the “GC clamps”.

The oligonucleotides were synthesized, ³²P-end labeled and subjected to an annealing reaction so that they were obtained in double-stranded form. They were incubated with wild-type p53 and mut p53, e.g. MethA p53 and Pro 273 p53, respectively, and subjected to an EMSA test. For this purpose, the following steps were taken:

Mixtures, which contained wild-type p53, MethA p53 and Pro 273 p53, respectively, were pre-incubated with 2 μg Poly dl:dC (non-specific competitor) in 10 mM hepes, pH 7.8; 50 mM KCl; 1 mM EDTA; 5 mM MgCl₂; 10% glycerin for 20 min.

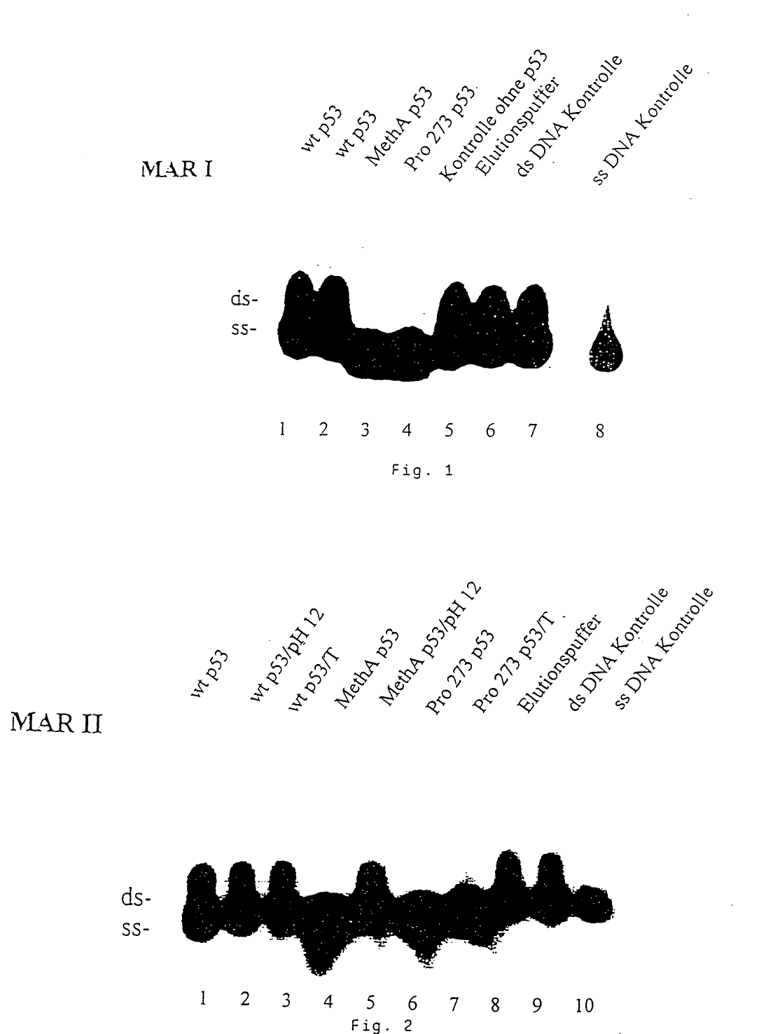

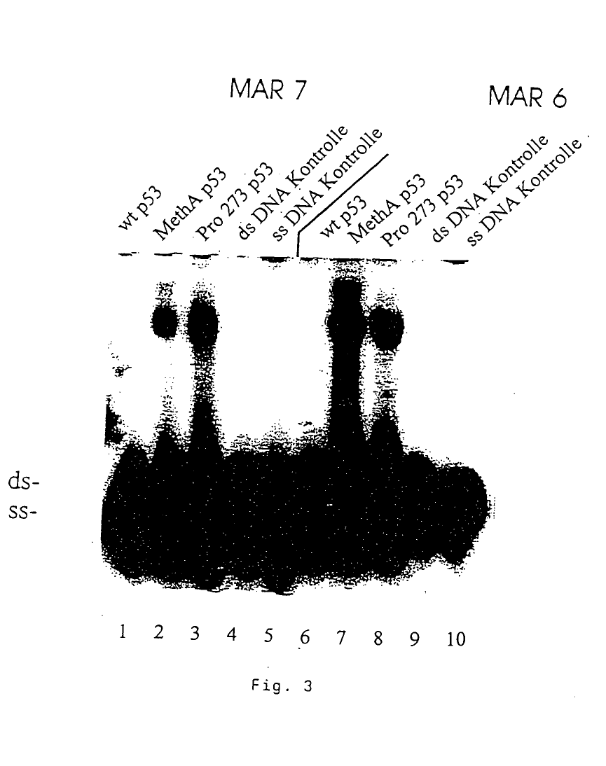

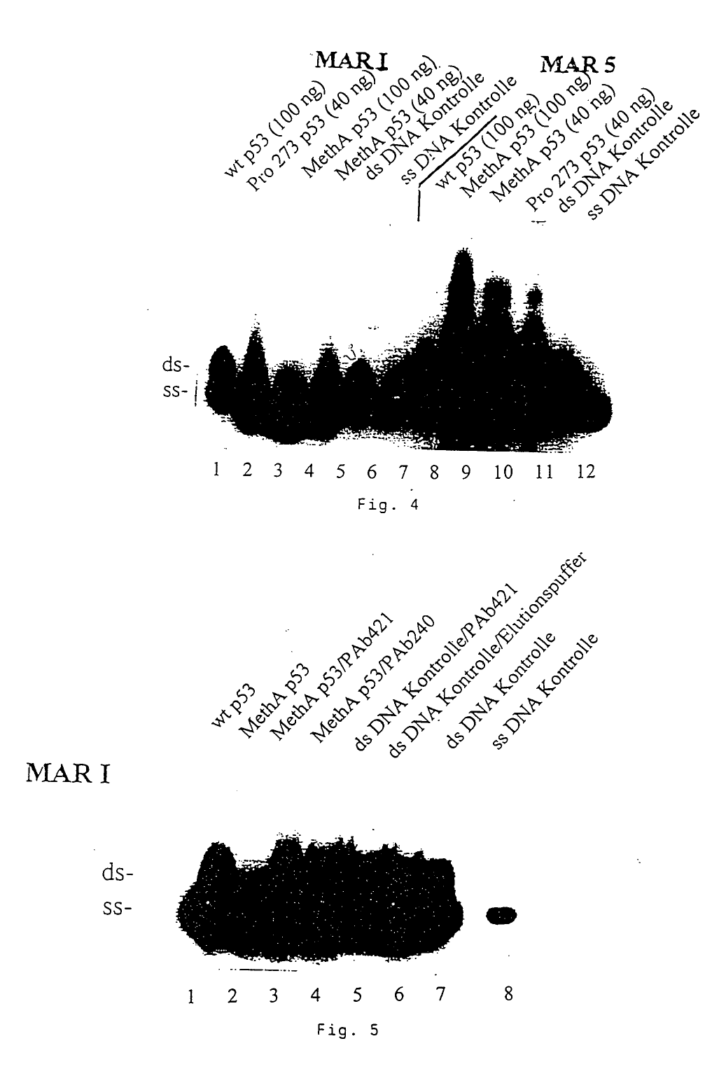

Following the pre-incubation, the labeled oligonucleotides were added and the mixtures were incubated at room temperature for 30 min. Thereafter, the mixtures were subjected to 4% native polyacrylamide gel electrophoresis for three hours before the gels were dried and autoradiographed (cf. FIGS. 1-4).

It showed that mut p53, e.g. MethA p53 and pro 273 p53, respectively, can cause a modulation of DNA, e.g. strand separation and complex formation, respectively, in the case of DNA with strand separating potential.

(b) Inhibition by DNA Strand Separation Caused by mut p53

The steps as described under item (a) were taken, with the exception that mixtures of MethA p53 also contained the antibodies PAb 421 and PAb 240, respectively. The oligonucleotides used were those which contained MARI DNA (cf. FIG. 5).

It showed that by a product inhibiting the mut p53 bonding to a DNA with strand separating potential, such as antibodies PAb 421 and PAb 240, respectively, the mut p53-induced DNA strand separation can be inhibited.

1. A method of inhibiting DNA modulation caused by mut p53, comprising the inhibition of mut p53 bonding to DNA with strand separating potential.

2. The method according to claim 1, wherein the modulation is a firm complex of mut p53 and the DNA with strand separating potential.

3. The method according to claim 1, wherein the modulation is a strand separation of the DNA with strand separating potential.

4. The method according to any claims 1 to 3, wherein mut p53 has a mutated core domain.

5. The method according to any of claims 1 to 3, wherein mut p53 has a mutated C terminus.

6. The method according to any of claims 1 to 5, wherein the DNA with strand separating potential comprises the sequence AATATATTT or a variation thereof.

7. The method according to claim 6, wherein the variation comprises the sequence AAAATATTT.

8. The method according to any of claims 1 to 7, wherein the DNA with strand separating potential is present in MAR DNA.

9. The method according to any of claims 1 to 4 and 6 to 8, wherein the inhibition takes place by a substance which inhibits a mutated core domain of p53.

10. The method according to claim 9, wherein the substance is the antibody PAb240.

11. The method according to any of claims 1 to 3 and 5 to 8, wherein the inhibition takes place by a substance which inhibits a mutated C terminus of p53.

12. The method according to claim 11, wherein the substance is the antibody PAb421.

13. The method according to any of claims 1 to 8, wherein the inhibition takes place by substances which inhibit a mutated core domain and a mutated C terminus of p53.

14. The method according to claim 13, wherein the substances are the antibodies PAb240 and PAb421.

15. The method according to any of claims 1 to 14, wherein DNA modulation caused by mut p53 is inhibited in the treatment of diseases.

16. The method according to claim 15, wherein the disease is a tumoral disease.

17. A system for identifying substances which are suitable for inhibiting DNA modulation caused by mut p53, comprising mut p53 and a DNA with strand separating potential.

18. The system according to claim 17, wherein the system also comprises a substance whose inhibitory effect on mut p53 bonding to a DNA with strand separating potential is tested.

19. The system according to claim 17 or 18, wherein mut p53 has a mutated core domain.

20. The system according to claim 17 or 18, wherein mut p53 has a mutated C terminus.

21. The system according to any or claims 17 to 20, wherein the DNA with strand separating potential is present in MAR DNA.

22. The system according to any of claims 17 to 21, wherein the DNA with strand separating potential comprises the sequence AATATATTT or a variation thereof.

23. The system according to claim 22, wherein the variation comprises the sequence AAAATATTT.


SEQUENCE LISTING (3 sequences)

SEQ ID NO: 1 - DNA, 35 bp, Artificial
  Synthetic
agtgtctttaatttctaatatatttagaaaactgc

SEQ ID NO: 2 - DNA, 36 bp, Artificial
  Synthetic
ttttaacaataataaattaagtttaaaatatttgcg

SEQ ID NO: 3 - DNA, 10 bp, Artificial
  Synthetic
aatataattt